(19) United States
(12) Patent Application Publication
(10) Pub. No.: US 2007/0231276 A1
(43) Pub. Date: Oct. 4, 2007

(54) STABLE DENTAL WHITENING COMPOSITIONS

(76) Inventors: Deepak Sharma, Plainsboro, NJ (US); Jenette Suh Edelstein, Belle Mead, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/689,000

(22) Filed: Mar. 21, 2007

Related U.S. Application Data
(60) Provisional application No. 60/787,896, filed on Mar. 31, 2006.

(51) Int. Cl. A61K 8/22

(52) U.S. Cl. 424/53; 424/49

(57) ABSTRACT

The invention is directed to a dental whitening composition including a dental whitening agent, a thickening agent, and a zwitterionic stabilizer in an amount effective to provide improved chemical and/or physical stability to the dental whitening composition.

### FIELD OF THE INVENTION

The present invention relates to whitening compositions for use in dental applications and that have improved chemical and/or physical stability.

### BACKGROUND OF THE INVENTION

Many dental products, both consumer and professional, are sold for the purpose of whitening teeth. For example, dental whitening strips having a whitening composition applied to a strip of material that is applied to the surface of teeth are known. Dental trays having whitening compositions contained therein also are applied to teeth for whitening of the teeth. The compositions used in such dental whitening products frequently include a gel that contains a whitening agent, such as hydrogen peroxide. The gels typically have a relatively high viscosity, so that they do not drip off of the teeth. The high viscosity is generally provided through the use of thickeners or gelling agents. However, peroxides typically break down gelling agents over time, leading to lower viscosity gels, which in turn may lead to products where the compositions do not maintain contact with teeth for the desired amount of time needed for efficacious whitening. In addition, hydrogen peroxide, as well as some other whitening agents, may be unstable over time in certain forms of dental whitening compositions. As such, the whitening efficacy of the compositions may be detrimentally affected over time, due to either or both of the reduced physical and/or chemical instability.

It thus is desirable to provide dental whitening compositions suitable for use in consumer or professional dental applications that have improved physical stability and/or chemical stability.

### SUMMARY OF THE INVENTION

The present invention provides a dental whitening composition suitable for use in whitening of teeth that includes a dental whitening agent, a thickening agent, and a zwitterionic stabilizer in an amount effective to provide increased physical and/or chemical stability to the dental whitening composition.

### DETAILED DESCRIPTION OF THE INVENTION

The compositions of the present invention include a whitening agent, preferably a non-abrasive whitening agent suitable for use in dental applications.

Suitable non-abrasive whitening agents known for use in dental applications include, but are not limited to, peroxides, metal chlorites, perborates, percarbonates, peroxyacids, and combinations thereof. Suitable peroxide compounds include hydrogen peroxide, calcium peroxide, carbamide peroxide, and mixtures thereof. Suitable metal chlorites include calcium chlorite, barium chlorite, magnesium chlorite, lithium chlorite, sodium chlorite, and potassium chlorite. Additional whitening agents that may be used include hypochlorite and chlorine dioxide. The whitening agent typically is present in an amount of from about 0.01% to about 40%, by weight, based on the total weight of the composition.

If a peroxide compound is chosen as the whitening agent, once incorporated into the composition, the peroxide compound should provide an amount of hydrogen peroxide equivalent to from about 0.1% to about 40%, for example from about 0.5% to about 20%, or from about 1% to about 10%, by weight, based on the total weight of the composition. To deliver this equivalent amount of hydrogen peroxide, the peroxide compound, for example hydrogen peroxide, is generally present in an amount from about 0.1% to about 40%, or from about 3% to about 20%, by weight, based on the total weight of the composition. For other peroxide compounds, slightly higher levels may be used to generate the equivalent weight of hydrogen peroxide in the composition.

The compositions of the present invention also contain a thickening agent. The thickening agent raises the viscosity of the composition sufficiently to prevent the composition from dripping or running off of teeth during the whitening process. Suitable thickeners include, but are not limited to carboxypolymethylene, carboxymethyl cellulose, carboxypropyl cellulose, poloxamer, carrageenan, Veegum, carboxyvinyl polymers, polyvinyl pyrrolidone, and natural gums such as gum karaya, xanthan gum, Guar gum, gum arabic, gum tragacanth, and mixtures thereof. The term “carboxypolymethylene” is used to denote a broad category of polymers, particularly copolymers of acrylic acid and polyallyl sucrose. Suitable carboxypolymethylene compositions may be obtained from B. F. Goodrich Company under the tradename “CARBOPOL”. Suitable Carbopol® thickeners include Carbopol® 934, 940, 941, 956, Carbopol® ETD 2001, ETD 2020, ETD 2050, Carbopol® Ultrez® 10 and mixtures thereof. Another tradename for carboxypolymethylene is CARBOMER. In its non-neutralized form, carboxypolymethylene can have a pH as low as 2.5.

The thickeners raise the viscosity of the compositions of the present invention to from about 200 to about 1,000,000 cps at low shear rates, for instance less than one reciprocal second (1/second). Preferably, the viscosity of the compositions of the present invention is from about 100,000 to about 800,000 cps and more preferably from about 400,000 to about 600,000 cps. The amount of thickening agent in the compositions of the present invention may range from about 0.1% to about 15%, for example from about 1% to about 10%, by weight, based on the total weight of the composition.

At least one zwitterionic stabilizer is added to the compositions of the present invention to provide improved physical and/or chemical stability to the composition compared to a similar composition that does not contain the zwitterionic stabilizer. While not intending to be limited by the following, in aqueous solutions at pH of between about 4.5 to about 7.5, certain molecules or compounds possess zwitterionic properties and possess functional groups that can act as a proton donor as well as an acceptor in hydrogen bonding interactions. Hence these stabilizers may increase hydrogen bonding in thickened gels of the present invention, rendering them more resistant to free radical initiated degradation. The zwitterionic stabilizer may stabilize the whitening agent, e.g. hydrogen peroxide, in gels utilizing a polymeric thickener, e.g. a Carbopol® thickener, by forming a complex but favorable network of hydrogen bonds with the peroxide and/or the polymer molecules. This enhanced and complex hydrogen bonding in these gels not only arrests the degradation of hydrogen peroxide and generation of hydroxyl radicals, which in turn attack thickening polymers, but also shield the site of radical attack on the thickening polymer. The stabilizer thus aids in preventing or minimizing degradation of the whitening agent and/or the polymer thickener over time. Such degradation is associated with the decrease of pH of the dental compositions over time. A decrease in the pH of the composition indicating degradation of the polymeric thickener is associated with a reduction in viscosity of the composition. Appreciable decreases in the viscosity of the compositions then result in compositions that may not remain in close contact of the teeth for a period of time necessary to provide effective whitening, thereby resulting in decreased whitening efficacy. In addition to whitening efficacy issues, a “runny” composition may create additional aesthetic issues affecting general consumer acceptance of the product.

Zwitterionic stabilizers useful in the present invention may be in the form of a molecule, a chemical compound, or mixtures thereof Suitable zwitterionic stabilizers include, but are not limited to, amino acids, short peptides, and their derivatives and such as glycine, di-glycine, tri-glycine, histidine, lysine, arginine, β-alanine, 4-amino butyric acid, and 6-amino hexanoic acid. Other classes of zwitterionic stabilizers include urea, betaine surfactants such as cocamidopropyl betaine, trimethylamine oxide, peptides, and combinations thereof. Selection of a suitable stabilizer may be made in view of the particular whitening agent and thickener being used, in addition to suitability for use in humans. Typically, longer chain compounds may be preferred for efficacy in that a greater distance between the respective positive and negative charges on the zwitterionic stabilizer may provide increased efficacy, although smaller compounds or molecules may be more preferable for use in humans. The zwitterionic stabilizer used in the compositions of the present invention is present in an amount effective to provide the physical and/or chemical stability to the composition. Typically, the amount of zwitterionic stabilizer may range from about 0.05% to about 5%, for example from about 0.2% to about 2%, by weight, based on total weight of the composition.

Compositions according to the present invention utilizing a zwitterionic stabilizer exhibit improved chemical and physical stability when compared to similar compositions that do not contain a zwitterionic stabilizer, even when conventional chelating agents such EDTA and/or citric acid are used. Gel compositions of the present invention typically retain at least about 75% of the formulated peroxide content when exposed to a temperature of about 40° C. over 8 weeks, or about 70% of peroxide content when exposed to 40° C. for about 12 weeks. In addition, pH of the gel compositions of the present invention typically are not reduced by more than about 25%, or not more than about 20%, when exposed to 40° C. for about 12 weeks. As noted herein above, a significant decrease in pH results in a significant reduction in viscosity of the gels of the present invention.

The addition of polyhydric solvents may further improve the chemical and/or physical stability of the compositions of the present invention. Suitable polyhydric solvents include, but are not limited to, polyvinyl alcohol, polyvinyl pyrrolidone, polyethylene glycol, polypropylene glycol, trehalose, lactose, sucrose, maltose, mannose, derivatives thereof, and mixtures thereof. When utilized, the amount of polyhydric solvent in the compositions of the present invention may range from about 0.1% to about 20%, for example from about 1% to about 10%, by weight, based on total weight of the composition.

The pH of dental whitening compositions is typically adjusted with strong bases, such as sodium hydroxide, potassium hydroxide, and the like. The final pH is typically from about 4 to about 7. With the compositions of the present invention, in addition to stabilizing the composition, the zwitterionic stabilizing compound may also serve as a buffering agent and help maintain pH under chemical stress. It may further allow use of less sodium hydroxide or potassium hydroxide to adjust the pH of the formula to, for example about 5.5.

An additional carrier material may also be added to the compositions of the present invention. Carrier materials can be humectants, pH-adjusting agents, non-zwitterionic stabilizing agents, desensitizing agents, and other components as listed below. Suitable non-zwitterionic stabilizing agents include benzoic acid, salicylic acid, butylated hydroxytoluene, tin salts, phosphates, and others. Suitable humectants include glycerin, sorbitol, polyethylene glycol, propylene glycol, and other edible polyhydric alcohols. Humectants may be present in an amount of from about 10% to about 95%, for example from about 50% to about 80% by weight, based on total weight of the composition. In addition to the above materials, a number of other carrier materials can also be added to the compositions of the present invention. Additional carriers include, but are not limited to, flavoring agents, sweetening agents such as saccharin, xylitol, opacifiers, coloring agents, and chelants such as ethylenediaminetetraacetic acid.

Desensitizing agents may also be used in the compositions of the present invention. These agents may be preferred for consumers who have sensitive teeth. Desensitizing agents include potassium nitrate, citric acid, citric acid salts, strontium chloride, amorphous calcium phosphate, and combinations thereof. Potassium nitrate is a preferred desensitizing agent. Other agents which provide the benefit of reduced tooth sensitivity may also be included in the compositions of the present invention. When used, the desensitizing agent will be present in an amount effective to reduce tooth sensitivity. Typically, this amount is from about 0.01% to about 10%, for example from about 0.1% to about 8%, or from about 1% to about 7%, by weight, based on total weight of the composition.

Anti-tartar agents may be incorporated into the compositions of the present invention. Suitable anti-tartar agents include phosphates, such as pyrophosphates, polyphosphates, polyphosphonates, and mixtures thereof. Pyrophosphate salts useful in the compositions include the dialkali metal pyrophosphate salts, tetra-alkali metal pyrophosphate salts, and mixtures thereof Suitable pyrophosphate salts include disodium dihydrogen pyrophosphate, tetrasodium pyrophosphate, tetrapotassium pyrophosphate, and mixtures thereof, in their unhydrated as well as hydrated forms. Anti-plaque agents may also be useful in the compositions of the present invention. Anti-plaque agents are any substances that inhibit the accumulation of bacterial deposits on the surfaces of the oral cavity. Examples include, without limitation, xylitol and other anti-microbial agents.

Fluoride ion sources, such as sodium fluoride or potassium fluoride, may also be useful in the compositions of the present invention. Fluoride ion is well known for use in oral care compositions as an anticaries agent.

The compositions of the present invention are prepared by standard techniques well known to those skilled in the art. If the composition comprises more than one phase, in general, the different phases will be prepared separately, with materials of similar phase partitioning being added in any order. The two phases will then be combined with vigorous stirring to form the multiphase system e.g., an emulsion or dispersion. Any ingredients in the formulation with high volatility, or which are susceptible to hydrolysis at high temperatures, will usually be added post mixing of the different phases with gentle stirring.

The compositions of the present invention may be utilized to whiten teeth by methods known in the art. For example, the compositions may be brushed onto the teeth with an applicator. Alternatively, the compositions may be applied onto teeth in the form of a coated strip. The compositions also may be utilized in multi-component dentifrice compositions. The use of conventional dental whitening trays is also suitable for use with the compositions of the present invention.

Examples are set forth below to further illustrate the nature of the invention and the manner of carrying it out. However, the invention should not be considered as being limited to the details thereof.

### EXAMPLE 1

Non-abrasive whitening gel compositions were prepared as follows. Water-soluble components were added with mixing into a non-reactive mixing vessel containing a portion of the formulation amount of water to form an aqueous pre-mix. The residual formulation amount of water was combined with humectants in a separate vessel and mixed. Thickener was then added slowly to the separate vessel to insure proper dispersion. Once all thickeners were wetted out, vacuum was applied to minimize aeration of the composition. Heat can be used to help the thickeners hydrate. The aqueous pre-mix was added to the separate vessel containing the composition along with the zwitterionic stabilizer, where applicable. Once all ingredients were dissolved and the temperature had cooled to a temperature of about 40° C. to about 50° C., the pH was adjusted as appropriate and the flavor and surfactants were added with mixing under vacuum. The whitening agent was then added to the vessel with mixing.

### EXAMPLE 1a

45.33% purified water, 30% glycerin, 17.14% hydrogen peroxide (35% solution), 0.12% sodium saccharin, 0.16% calcium disodium EDTA, 1.8% Carbomer (CARBOPOL Ultrez 10), 3% PEG 400 hydrogenated castor oil, 0.2% L-Menthol, 1% wintergreen mint flavor, 0.8% glycine, and 0.45% potassium hydroxide (to pH 5.5).

### EXAMPLE 1b

46.13% purified water, 30% glycerin, 17.14% hydrogen peroxide (35% solution), 0.12% sodium saccharin, 0.16% calcium disodium EDTA, 1.8% Carbomer (CARBOPOL Ultrez 10), 3% PEG 400 hydrogenated castor oil, 0.2% L-Menthol, 1% wintergreen mint flavor, and 0.45% potassium hydroxide (to pH 5.5).

Samples 1a and 1b were stored in capped centrifuge tubes at 40° C. Samples 1a and 1b were analyzed at various points in time for pH and hydrogen peroxide content. A significant drop in pH is associated with an undesirable decrease in viscosity of the composition. The results of the pH measurements are shown in Table 1. The results of the hydrogen peroxide content measurements are shown in Table 2.

TABLE 1

| | pH | | | | |
|---|---|---|---|---|---|
| Sample | Initial | 2 week | 4 weeks | 8 weeks | 12 weeks |
| 1a (40° C.) | 5.2 | 4.9 | 4.7 | 4.5 | 4.3 |
| 1b (40° C.) | 5.3 | 5.0 | 4.2 | 3.6 | 3.0 |

NA = not analyzed.

The data in Table 1 demonstrates that samples containing a zwitterionic stabilizer, e.g. glycine, maintain pH for a longer period of time than samples without the zwitterionic stabilizer. As a decrease in pH in such gels is associated with a loss of viscosity, the samples containing the zwitterionic stabilizer are more physically stable than the samples without the zwitterionic stabilizer.

TABLE 2

| | Percent Hydrogen Peroxide | | | | |
|---|---|---|---|---|---|
| Sample | Initial | 1 week | 4 weeks | 6 weeks | 8 weeks |
| 1a (40° C.) | 4.0 | NA | 3.7 | NA | 3.6 |
| 1b (40° C.) | 4.0 | 4.0 | 3.7 | 0.4 | 0.1 |

NA = not analyzed.

The data in Table 2 demonstrates that samples containing a zwitterionic stabilizer, e.g. glycine, maintain the level of hydrogen peroxide initially formulated in the composition for a longer period of time than samples without the zwitterionic stabilizer. Samples containing the zwitterionic stabilizer are therefore more chemically stable than the samples without the zwitterionic stabilizer.

### EXAMPLE 2

Three gel compositions were prepared according to the procedure described above to evaluate the effect of a zwitterionic stabilizer in combination with different chelating agent such as EDTA and citric acid. Gels were formulated as noted below, with 4% by weight of hydrogen peroxide level and 0.8% by weight of glycine. EDTA or citric acid was present as indicated.

| Ingredient | Weight % |
|---|---|
| Glycerin, 99.5% USP, Glycon G100 | 50.94 |
| Water, Purified | 22.64 |
| Water, Purified | 2.5 |
| Carbopol 974P | 1.2 |
| Hydrogen Peroxide Concentrate (35%, Peralkali) | 11.43 |
| Glycine, USP | 0.8 |
| Citric Acid or EDTA | 0.5 |
| SLS Texapon | 0.3 |
| Noville Flavor AN149839 | 0.35 |
| Polyethylene Glycol 400 - NF, Carbowax Sentry | 8.0 |
| Potassium Hydroxide | 0.8 |
| Sodium Saccharin, USP, Syncal S | 0.3 |
| Sodium Fluoride, USP | 0.24 |
| | 100.00 |

The samples were stored at 40° C. and monitored for peroxide content and pH over 13 weeks. The percent formulated peroxide retention (FPR) and pH reduction data is presented in Table 3. As can be seen, the gel without the zwitterionic stabilizer, e.g. glycine, was susceptible to chemical (H₂O₂) and physical degradation (pH) over time under temperature stress of 40° C., despite the presence of EDTA. However, addition of the glycine stabilized the gel compositions by inhibiting reduction in pH, which would result in a reduction in gel viscosity, as well as maintaining a significant portion of the formulated peroxide content, thereby preserving the whitening efficacy of the gel.

TABLE 3

| | Glycine + EDTA | | | Glycine + Citric acid | | | EDTA without glycine | | |
|---|---|---|---|---|---|---|---|---|---|
| Wks | % H₂O₂ | % FPR | pH | % H₂O₂ | % FPR | pH | % H₂O₂ | % FPR | pH |
| 0 | 4.00 | 100.00 | 4.96 | 3.94 | 100.00 | 4.89 | 3.9 | 100 | 4.795 |
| 1 | 3.45 | 86.35 | 4.82 | 3.81 | 96.70 | 4.86 | 3.6332 | 93.16 | 4.792 |
| 2 | 3.42 | 85.52 | 4.88 | 3.75 | 95.18 | 4.94 | 3.3306 | 85.40 | 4.744 |
| 3 | 3.35 | 83.84 | 4.99 | 3.72 | 94.42 | 4.99 | 2.9426 | 75.45 | 4.4 |
| 4 | 3.32 | 83.01 | 5.00 | 3.69 | 93.65 | 4.95 | 2.4746 | 63.45 | 4.1 |
| 5 | 3.28 | 81.89 | 4.91 | 3.61 | 91.62 | 4.99 | 2.0865 | 53.50 | 3.7 |
| 6 | 3.20 | 79.94 | 4.87 | 3.58 | 90.86 | 4.97 | 1.7328 | 44.43 | 3.4 |
| 7 | 3.25 | 81.34 | 4.72 | 3.53 | 89.59 | 4.89 | 1.4344 | 36.78 | 3.4 |
| 9 | 3.03 | 75.77 | 4.70 | 3.38 | 85.79 | 4.85 | 1.2987 | 33.30 | 3.2 |
| 10 | 2.63 | 65.74 | 4.63 | 3.37 | 85.53 | 4.52 | 1.092 | 28.00 | 3.1 |
| 12 | 2.94 | 73.54 | 4.45 | 3.20 | 81.22 | 4.66 | 0.936 | 24.00 | 3 |
| 13 | 2.89 | 72.14 | 4.40 | 3.10 | 78.68 | 4.40 | 0.702 | 18.00 | 2.9 |

We claim:

1. A dental whitening composition, comprising:
a dental whitening agent;
a thickening agent; and a
zwitterionic stabilizer in an amount effective to provide physical and/or chemical stability to the dental whitening composition.

2. The composition according to claim 1 wherein the thickening agent is 10 selected from the group consisting of carboxypolymethylene, carboxymethyl cellulose, carboxypropyl cellulose, poloxamer, carrageenan, Veegum, carboxyvinyl polymers, gum karaya, xanthan gum, Guar gum, gum arabic and gum tragacanth.

3. The composition according to claim 1 wherein the whitening agent is selected from the group consisting of peroxides, metal chlorites, perborates and percarbonates.

4. The composition according to claim 1 wherein the whitening agent is selected from the group consisting of hydrogen peroxide, calcium peroxide and carbamide peroxide.

5. The composition according to claim 1 wherein the zwitterionic stabilizer is selected from the group consisting of glysine, lysine, arginine, β-alanine, 4-amino butyric acid, 6-amino hexanoic acid, urea and trimethylamine oxide.

6. The composition of claim 5 comprising from about 0.5% to about 5% of the zwitterionic stabilizer based on total weight of the composition

7. The composition of claim 6 wherein the zwitterionic stabilizer comprises glycine.

8. The composition of claim 7 wherein the whitening agent comprises a peroxide and the thickening agent comprises carboxypolymethylene.

9. The composition of claim 8 wherein the composition comprises from about 0.1% to about 40% of hydrogen peroxide as the whitening agent and from about 0.1% to about 15% of the carboxypolymethylene, based on total weight of the composition.

10. The composition of claim 1 wherein the effective amount of zwitterionic stabilizer is effective to retain at least about 75% of formulated peroxide content in the composition after exposure for 8 weeks at 40° C.

11. The composition of claim 1 wherein the effective amount of zwitterionic stabilizer is effective to retain at least about 70% of formulated peroxide content in the composition after exposure for 8 weeks at 40° C.

12. The composition of claim 1 wherein the effective amount of zwitterionic stabilizer is effective to prevent reduction of the pH of the composition by more than about 25% after exposure for 12 weeks at 40° C.

13. The composition of claim 1 wherein the effective amount of zwitterionic stabilizer is effective to prevent reduction of the pH of the composition by more than about 20% after exposure for 12 weeks at 40° C.
